SEQUENCE LISTING (17 sequences)

SEQ ID NO: 1 - protein, 52 aa, Artificial Sequence
  synthetic fragment of human fibroblast growth factor-2, human basic FGF, heparin-binding growth factor, prostatropin (FGF2, hFGF-2, hbFGF)
EERGVVSIKGVCANRYLAMKEDGRLLASKCVTDECFFFERLESNNYNTYRSR

SEQ ID NO: 2 - protein, 43 aa, Artificial Sequence
  synthetic fragment of human fibroblast growth factor-2, human basic FGF, heparin-binding growth factor, prostatropin (FGF2, hFGF-2, hbFGF)
GVCANRYLAMKEDGRLLASKCVTDECFFFERLESNNYNTYRSR

SEQ ID NO: 3 - protein, 32 aa, Artificial Sequence
  synthetic fragment of human fibroblast growth factor-2, human basic FGF, heparin-binding growth factor, prostatropin (FGF2, hFGF-2, hbFGF)
EDGRLLASKCVTDECFFFERLESNNYNTYRSR

SEQ ID NO: 4 - protein, 28 aa, Artificial Sequence
  synthetic fragment of human fibroblast growth factor-2, human basic FGF, heparin-binding growth factor, prostatropin (FGF2, hFGF-2, hbFGF)
LLASKCVTDECFFFERLESNNYNTYRSR

SEQ ID NO: 5 - protein, 24 aa, Artificial Sequence
  synthetic fragment of human fibroblast growth factor-2, human basic FGF, heparin-binding growth factor, prostatropin (FGF2, hFGF-2, hbFGF)
KCVTDECFFFERLESNNYNTYRSR

SEQ ID NO: 6 - protein, 15 aa, Artificial Sequence
  synthetic fragment of human fibroblast growth factor-2, human basic FGF, heparin-binding growth factor, prostatropin (FGF2, hFGF-2, hbFGF)
FERLESNNYNTYRSR

SEQ ID NO: 7 - protein, 155 aa, Homo sapiens [sequence omitted]

SEQ ID NO: 8 - DNA, 27 bp, Artificial Sequence
  synthetic PCR amplification primer hbFGF full length sense
ggaattcccatggcagccgggagcatc

SEQ ID NO: 9 - DNA, 27 bp, Artificial Sequence
  synthetic PCR amplification primer hbFGF pep1 sense
ggaattcccgaagagagaggagttgtg

SEQ ID NO: 10 - DNA, 27 bp, Artificial Sequence
  synthetic PCR amplification primer hbFGF pep2 sense
ggaattcccgtgtgtgctaaccgttac

SEQ ID NO: 11 - DNA, 45 bp, Artificial Sequence
  synthetic PCR amplification primer hbFGF full length antisense
ctcgagtcaatgatgatgatgatgatggctcttagcagacattgg

SEQ ID NO: 12 - DNA, 45 bp, Artificial Sequence
  synthetic PCR amplification primer hbFGF pep antisense
ctcgagtcaatgatgatgatgatgatgccttgaccggtaagtatt

SEQ ID NO: 13 - protein, 37 aa, Artificial Sequence
  synthetic peptide pep 3 (37 aa), peptide #3, pep3 with C-terminus 5x HIS-tag
EDGRLLASKCVTDECFFFERLESNNYNTYRSRHHHHH

SEQ ID NO: 14 - protein, 29 aa, Artificial Sequence
  synthetic peptide pep 4 (29 aa), peptide #4, pep4 with C-terminus 5x HIS-tag
KCVTDECFFFERLESNNYNTYRSRHHHHH

SEQ ID NO: 15 - protein, 20 aa, Artificial Sequence
  synthetic peptide pep 5 (20 aa), peptide #5, pep5 with C-terminus 5x HIS-tag
FERLESNNYNTYRSRHHHHH

SEQ ID NO: 16 - protein, 6 aa, Artificial Sequence
  synthetic C-terminus 6x HIS-tag, HIS-tag (6x)
HHHHHH

SEQ ID NO: 17 - protein, 5 aa, Artificial Sequence
  synthetic C-terminus 5x HIS-tag
HHHHH

(19) United States
(12) Patent Application Publication
(10) Pub. No.: US 2012/0316113 A1
(43) Pub. Date: Dec. 13, 2012

(54) FGF BASED FIBRIN BINDING PEPTIDES

(75) Inventors: Heinz Redl, Vienna (AT); Tatjana Morton, Vienna (AT)

(73) Assignees: Baxter Healthcare S.A., Glattpark (Opfikon) (CH); Baxter International Inc., Deerfield, IL (US)

(21) Appl. No.: 13/462,624

(22) Filed: May 2, 2012

Related U.S. Application Data
(60) Provisional application No. 61/481,337, filed on May 2, 2011.

(51) Int. Cl. A61K 38/18; C07K 7/08; A61P 43/00; C07K 14/50

(52) U.S. Cl. 514/9.1; 530/324; 530/326; 530/350; 530/325

(57) ABSTRACT

The present invention relates to a polypeptide fragments of Fibroblast Growth Factor 2, wherein said fragments are adapted to bind to fibrinogen or to fibrin and/or increase cell proliferation, differentiation or migration. These fragments are suitable for promoting wound healing and/or hemostasis.

### CROSS-REFERENCES TO RELATED APPLICATIONS

This application is a nonprovisional of, and claims the benefit of priority to, U.S. provisional patent application No. 61/481,337 filed May 2, 2011, the content of which is incorporated herein by reference.

### FIELD OF THE INVENTION

The present invention relates to peptides having a fibrin(ogen) binding domain and with biological activity on various cell types.

### BACKGROUND OF THE INVENTION

Fibrin is naturally associated with a number of growth factors that bind to fibrinogen and may promote wound healing (1;2). Such growth factors attract or stimulate cells involved in tissue repair (3;4). Human Fibroblast growth factor-2 (hFGF-2) holds significant potential as a therapeutic additive to sealant products, because hFGF-2 can stimulate wound healing, tissue regeneration, and angiogenesis.

Human FGF-2 (human basic FGF, heparin-binding growth factor or prostatropin) belongs to a family of heparin-binding growth factors (5). As a single-chain protein, hFGF-2 exists in variants of 146 amino acids, an isoelectric point of 9.6 and a molecular weight of about 17,400 Dalton (6;7). The human FGF-2 gene is expressed in bone marrow, lymph node, pancreas, thymus and presumably spleen (4;8). Human FGF-2 was characterized by Florkiewicz and Sommer and is the prototype member of the FGF family (9). It does not contain any disulfide bonds and is not glycosylated (6). The structure of hFGF-2, revealed by X-ray crystallography, shows a similar folding structure as seen with Interleukin (IL-1) (4;10).

Human FGF-2 is a multifunctional protein with a wide spectrum of biological activities. Human FGF-2 stimulates a variety of physiological processes, including cell proliferation, cell differentiation and cell migration (11). More recently, hFGF-2 has been further recognised as a hematopoietic cytokine and may play an important role in wound healing and angiogenesis (3). It is released after tissue injury and during inflammatory processes, and also during the proliferation of tumor cells (12).

The high-affinity and saturable binding of hFGF-2 to fibrinogen and fibrin, indicates an important level of influence at sites of injury between growth factors with critical cell regulatory functions and the fibrin matrix (13).

Fibrin is a fibrous protein, and occurs physiologically in the final step of the blood clotting cascade to form a hemostatic plug or clot over a vessel lesion. Fibrinogen reacts with activated thrombin and builds a soluble fibrin monomer network. These monomers are crosslinked to an insoluble fibrin network by the transglutaminase FXIII or fibrin stabilizing factor. The application of fibrin gel is widely-used in hemostasis and tissue sealing due to its high biological tolerance and its naturally proteolytic resorption (14-16). Therefore, fibrin gels are perfectly suited as a matrix, or depot for living cells and bioactive agents acting as a slow release delivery system (13;17). Due to the hydrated and porous structure of fibrin, however, substances without specific affinity to fibrin will exit the matrix by diffusion relatively fast. In comparison, natural fibrin binding proteins, or binding sequences thereof, can be linked to target substances without a natural binding affinity to fibrin, thereby retaining them in the fibrin matrix (18). Natural fibrin binding proteins as growth factors, e.g. vascular endothelial growth factor-165 (VEGF-165), interleukin-1 beta (IL-1β) and hFGF-2, show a high binding affinity to fibrinogen and fibrin and therefore lead to a continuous slow release oat of the fibrin matrix (1-4).

The U.S. Pat. No. 6,713,453 describes the use of VEGF-165 fragments comprising a fibrin binding domain in a conjugate with a pharmaceutically active substance to localize this substance to the site of fibrin clots. Such substances are e.g. factors that improve regenerative processes, like growth factors and antibiotics.

It is a goal of the present invention to provide fibrin binding substances to facilitate the localization of pharmaceutically active substances to fibrin and/or lead to fibrin(ogen)-fibrin(ogen) and/or tissue interactions. It is a further goal of the invention to provide substances that bind fibrin and have biological activity themselves.

### BRIEF SUMMARY OF THE INVENTION

These objects are solved by a polypeptide fragment of Fibroblast Growth Factor 2, wherein said fragment binds or is adapted to bind to fibrinogen or to fibrin and/or increases cell proliferation, differentiation or migration.

The fragments of the present invention can facilitate binding to fibrinogen, fibrin or to both, in particular in fibrin clots. A bound polypeptide to fibrin or fibrinogen can also be provided in pharmaceutical preparations like fibrinogen/fibrin gets. In case of di or multimeric peptides, this can also lead to gel forming without thrombin action or, when carrying a reactive group, better adhesion to tissue. At the same time, the polypeptide may provide biological activity that increases cell proliferation, differentiation and/or migration. This activity can be useful for increasing cellular regeneration of injured tissues, in particular for wound healing applications and/or hemostasis. Due to binding to fibrin or fibrinogen, the polypeptide fragment is bound sufficiently to the fibrin matrix so that elution of die fragment is not possible by simple diffusion, but mainly dependent on the affinity of the fragment to fibrin or fibrinogen. In addition the described peptides can carry a tissue reactive group in the non-binding part of the peptide such as —NHS—, or other reactive chemical groups (e.g. —SH) or collagen binding sequences as described (LIT), antibodies or antibody fragments to tissue, components (e.g. extracellular matrix or cell membrane molecules).

The polypeptide fragment can be conjugated to further pharmaceutical active substances as e.g. disclosed in the U.S. Pat. No. 6,713,453 (incorporated herein by reference) to further increase or modify the biological activity of the conjugate. Binding of the polypeptide fragment with a substance can be directly, covalently, such as by expression of a fusion protein or mediated by a chemical linking agent. Such conjugates are adapted to bind fibrin and/or fibrinogen and/or to increase cell proliferation, differentiation or migration by the inventive polypeptide fragment. The pharmaceutically active substance may further increase such effects.

The term “adapted to bind to fibrinogen or to fibrin” relates to the ability to bind either to fibrin or to fibrinogen, or to both fibrinogen and fibrin. If the binding capacity is for both fibrin, and fibrinogen, it is possible to form a fibrin gel with fibrinogen molecules which are already “loaded” with the polypeptide fragment and, optionally, any attached or conjugated pharmaceutically active substances, to allow a homogenous deposition of the polypeptide fragment and distribution throughout a fibrin matrix created by the fibrin gel.

The inventive polypeptides or conjugates are particularly suitable to improve wound healing and/or hemostasis in a subject, or for inducing cell migration to a fibrin clot or to induce cell differentiation or proliferation at the location of a fibrin clot, especially of cellular effects involved in wound healing and/or hemostasis. In a dipeptide or multimeric form they can also provide sealing by forming a film and potential hemostasis. Uses of the polypeptides for these purposes form a special aspect of the present invention.

In one aspect, embodiments of the present invention encompass a polypeptide fragment of Fibroblast Growth Factor 2. In some cases, a fragment may bind to fibrinogen. In some cases, a fragment may bind to fibrin. In some cases, a fragment may increase cell proliferation, differentiation, or migration. In some cases, a fragment may promote wound healing. In some cases, a fragment may be conjugated to a pharmaceutically active substance. In some cases, a fragment may be conjugated, to a pharmaceutically active substance by a linking agent. Relatedly, a linking agent can be selected from the group of carbodiimides, such as 1-ethyl-3-(3-dimethyl-aminopropyl)carbodiimid (EDC), bis-diaxotized benzidine (BDB), a bifunctional glutaraldehyde, a heterobifunctional reagent such as m-maleimidobenzoyl-N-hydroxysuccinimide ester (MBS), SH coupling.

In some cases, a polypeptide fragment can have essentially the biological activity as fibroblast growth factor 2 on cell proliferation, differentiation or migration, hi some cases, a polypeptide fragment can increase proliferation of a fibroblast cell, a myoblast cell, an endothelial cell, a stem cell, or any combination thereof.

In some cases, a polypeptide can include an amino acid sequence of EERGVVSIKG VCANRYLAMK EDGRLLASKC VTDECFFFER LESNNYNTYR SR (SEQ ID NO: 1). In some eases, a polypeptide can include an amino acid sequence of GVCANRYLAM KEDGRLLASK CVTDECFFFE RLESNNYNTY RSR (SEQ ID NO:2). In some cases, a polypeptide can include an amino acid sequence of EDGRLLASKC VTDECFFFER LESNNYNTYR SR (SEQ ID NO:3). In some cases, a polypeptide can include an amino acid sequence of LLASKCVTDE CFFFERLESN NYNTYRSR (SEQ ID NO:4). In some cases, a polypeptide can include an amino acid sequence of KCYTDECFFF ERLESNNYNT YRSR (SEQ ID NO:5). In some cases, a polypeptide can include an amino acid sequence of FERLESNNYN TYRSR (SEQ ID NO:6). According to some embodiments, with any of the above polypeptides, there may optionally be 1 to 10 amino acid substitutions or deletions. In some instances, the substitutions are conserved substitutions.

In some polypeptide fragment embodiments, the Fibroblast Growth Factor 2 is a human Fibroblast Growth Factor 2. In some polypeptide fragment embodiments, the Fibroblast Growth Factor 2 may include the amino acid sequence

(SEQ ID NO: 7)
MAAGSITTLPALPEDGGSGAFPPGHFKDPKRLYCKNGGFFLRIHPDGRV
DGVREKSDPHIKLQLQAEERGVVSIKGVCANRYLAMKEDGRLLASKCVT
DECFFFERLESNNYNTYRSRKY TSWYVALKRTGQYKLGSKTGPGQKAI
LFLPMSAKS.

In some instances, a polypeptide fragment can have a length of at least 20 amino acids. In some instances, a polypeptide fragment can have a length of up to 140 amino acids. In some instances, a polypeptide fragment may include a label.

In some instances, a polypeptide fragment may be linked to a protease inhibitor or a lysine derivative. Relatedly, in some instances the protease inhibitor may increase stability of fibrin or prevents fibrin degradation. In some instances, the protease inhibitor may be aprotinin or eglin.

In some instances, a polypeptide fragment, may be linked to a tissue binding reacting sequence. In some eases, a polypeptide fragment may be adapted to crosslink fibrinogen or fibrin chains. In some cases, a polypeptide fragment may be used in a therapy. In some cases, the therapy may include a blood clotting reaction. In some cases, the therapy may include a fibrin aggregation reaction. In some cases, the therapy may include a fibrinogen aggregation reaction. In some eases, the therapy may include a fibrin, solidification reaction. In some cases, the therapy may include a fibrinogen solidification reaction. In some eases, the therapy may include wound healing. In some eases, the therapy may include hemostasis.

In another aspect, embodiments of the present invention encompass a pharmaceutical, composition that includes a polypeptide fragment as described herein. In some cases, a pharmaceutical composition may include a buffer, a stabilizer, a protease inhibitor, or a carrier, or any combination thereof. In some cases, a pharmaceutical composition may include fibri. In some cases, a pharmaceutical composition may include fibrinogen.

In still a further aspect, a polypeptide fragment as described herein can be used as linker between fibrin and a pharmaceutically active substance.

In another aspect, embodiments of the present invention encompass methods of improving wound healing in a subject that include administering a polypeptide fragment as disclosed herein to the subject. In another aspect, embodiments of the present invention encompass methods of improving hemostasis in a subject that include administering a polypeptide fragment as disclosed herein to the subject.

In another aspect, embodiments of the present invention encompass methods of inducing cell migration to a fibrin clot or cell differentiation or proliferation at the location of a fibrin clot. Exemplary methods may include administering a polypeptide as disclosed herein to the clot. In a further aspect, embodiments of the present invention encompass methods of localizing a pharmaceutically active substance to a fibrin clot. Exemplary methods may include administering a conjugate of a polypeptide as disclosed herein with the pharmaceutically active substance to the clot. In another aspect, embodiments of the present invention encompass a DNA molecule that encodes a polypeptide as disclosed herein. In another aspect, embodiments of the present invention also encompass kits that include a polypeptide as disclosed herein. In some cases, a kit may include an amount of fibrinogen. In some cases, a kit may include an amount of a pharmaceutically active substance.

For a fuller understanding of the nature and advantages of the present invention, reference should be had to the ensuing detailed description taken in conjunction with the accompanying drawings.

### BRIEF DESCRIPTION OF THE DRAWINGS

The patent or application contains at least one drawing executed in color. Copies of this patent or patent application publication with color drawing(s) will be provided by the Office upon request and payment of the necessary fee.

FIG. 1 shows a scheme of the truncated FGF-2 fragments/peptides in comparison to full length human FGF-2.

FIG. 2 shows the 3-dimensional structure of human FGF-2 (˜17.4 kD protein) The red part (bounded by arrows A and B) identifies the binding domain (15 bp) of FGF-2 on fibrinogen (plotted using Visual Molecular Dynamics)

FIG. 3 shows the expression and purification of FGF-2 peptides, (a) The expression of fused GST-pep1 (˜32 kD), GST-pep2 (˜31 kD), and GST-pep3 (˜29 kD) was analysed by Western blot technique developed with a monoclonal anti-6His-peroxidase antibody. Lane 1, 3, 5 supernatant; lane 2, 4, 6 inclusion bodies.

(b) The cleaved and purified pep1 (˜6.2 kD), pep2 (˜5.2 kD), and pep3 (˜4.0 kD) were continued by discontinuous tricine SDS-PAGE stained with Coomassie brilliant blue.

FIG. 4 shows the chemically synthesized peptides pep3 (˜4.0 kD), pep4 (˜3.1 kD), and pep5 (˜2.1 kD) detected by discontinuous tricine SDS-PAGE stained with Coomassie brilliant blue.

FIG. 5 shows the fibrin(ogen) binding assay with different concentrations of FGF-2 and FGF-2 peptides (pep1-pep5). (a) The graph represents the amount of bound purified (FGF-2, pep1, and pep2) respectively synthesized (pep3, pep4, and pep5) FGF-2 and peptides to fibrinogen, (b) The graph demonstrates the amount of bound FGF-2 and peptides to fibrin. The values are expressed as mean±SD (*p<0.01; n=6).

FIG. 6 shows the fitted association curves of the sensorgrams of recombinant full-length hFGF-2 (102 aa) compared to the FGF-2 peptides pep1 (68 aa), pep2 (48 aa), pep3 (37 aa), pep4 (28 aa) and pep5 (20 aa). The sample pep4 showed similar binding characteristics as full-length hFGF-2. The values are expressed as mean±SD (n=3).

FIG. 7 shows the viability assay of mouse myoblast (C2C12) cells incubated with different concentrations of FGF-2 and FGF-2 peptides (pep1-pep5). The values are expressed as mean±SD (*p<0.01; n=6).

FIG. 8a shows the scheme for binding of aprotinin to fibrin via a covalently bound FS-anchor as rhFGF-2, His-FGF-2, pep1 (68 aa), pep3 (37 aa) and pep4 (28 aa) and PBS as a control.

FIG. 8b shows the observed results of the stability level of the fibrin clots with covalently bound, aprotinin through different FS-anchors on day 5.

### DETAILED DESCRIPTION OF THE INVENTION

The present invention provides small peptides derived from human Fibroblast Growth Factor-2 (hFGF-2) that have a binding affinity to fibrin and, preferably, also to fibrinogen, and exert biological activity, in particular to promote regenerative processes. The fibrin and fibrinogen, binding domain of hFGF-2 is located in the 3^{rd} quarter of the amino acid sequence of hFGF-2. The present invention is based on the thorough selection of peptides that both maintain the fibrin/fibrinogen binding activity of hFGF-2 and also can positively affect cell proliferation, differentiation or migration associated with wound healing and/or hemostasis. In particular, it was found that the domain for biological activity and for binding to fibrin or fibrinogen is located on the same segment of the hFGF-2 sequence. Furthermore, it was found that small fragments can be obtained that maintain both activities while benefiting from advantages associated with fragments such as increased stability and reduced degradation.

The inventive polypeptide is a fragment of FGF-2. As used, herein a “fragment” relates to a part of a sequence and excludes the whole length sequence.

The inventive polypeptide fragments have biological activity which includes an increased cell proliferation, differentiation or migration. The activity in particular includes promotion of cell migration into wound areas, initiation of new blood vessel formation, the stimulation or suppression of extracellular matrix production and/or increase of cell survival important in a healing process. In particular, the polypeptide may promote wound healing and/or hemostasis.

In special embodiments, the polypeptide has essentially the biological activity as fibroblast growth factor 2 on cell proliferation, differentiation, or migration. “Essentially the biological activity” herein may refer to a biological activity of the same magnitude, in particular within the range of 1% or more of the biological activity of FGF-2. In preferred embodiments, the activity is at least 1%, 2%, 4%, 5%, 6%, 8%, 10%, 15%, 20%, 25%, 30%, 40%, 50% or more of FGF-2. The biological activity on cell proliferation, differentiation or migration can be assessed by standard assays such as by using mouse myoblast cell lines (e.g. cell line C2C12), mouse fibroblast cell lines (e.g. cell MC3T3), isolated human umbilical vein endothelial cells (e.g. huVEC), isolated human adipose derived stem cells. Such assays are e.g. disclosed in the example section. The hFGF-2 peptide fragments of the present invention may stimulate the up-regulation in human adipose derived stem cells of essential extracellular matrix proteins and cytoskeletal elements, which are key patterns for tendon and ligament development.

In particular preferred embodiments of the present invention the polypeptide increases proliferation of a fibroblast cell, a myoblast cell, an endothelial cell, a stem cell or any combination thereof. In particular, the cells may be involved in regenerative processes, preferably in wound healing and/or hemostasis.

In particular preferred embodiments the inventive polypeptide is adapted to cross-link fibrinogen or fibrin chains. The possibility and ability to cross-link fibrinogen or fibrin chains, in particular in fibrin clots, further enhances stability and increases wound resistance to internal or external disturbances.

By binding to fibrin, the inventive fragment may increase fibrin stability and/or prevent fibrin degradation. In further variation the binding of a di- or multimeric human FGF derived peptide may lead to gel formation.

In preferred embodiments, the inventive polypeptide fragment, comprises an amino acid sequence of

(SEQ ID NO: 1)
EERGVVSIKG VCANRYLAMK EDGRLLASKC VTDECFFFER
LESNNYNTYR SR,
(SEQ ID NO: 2)
GVCANRYLAM KEDGRLLASK CVTDECFFFE RLESNNYNTY RSR,
(SEQ ID NO: 3)
EDGRLLASKC VTDECFFFER LESNNYNTYR SR,
(SEQ ID NO: 4)
LLASKCVTDE CFFFERLESN NYNTYRSR,
(SEQ ID NO: 5)
KCVTDECFFF ERLESNNYNT YRSR,
or
(SEQ ID NO: 6)

FERLESNNYN TYRSR

with optionally one to ten amino acid substitutions or deletions.

As used herein, “comprising” shall be understood in an open-ended sense in the meaning that the peptide at a minimum contains these sequences, but may comprise further amino acids. In particular preferred embodiments, the inventive fragment may comprise at least or at most 1, 5, 1.0, 20, 30, 40, 50 or 60 amino acid additions.

In specific embodiments of the present invention, the amino acid fragments may comprise optionally 1 to 10 amino acid substitutions or deletions. The fragment may comprise 1, 2, 3, 4, 5, 6, 7, 8, 9 or 10 amino acid substitutions. In particular preferred embodiments, the substitutions increase stability, such as by introducing further cysteine residues.

In preferred embodiments the substitutions are conserved substitutions. Conserved substitutions are mutations within an amino acid group. Amino acids are usually grouped according to their polarity, charge and/or size. The following groups are noteworthy: basic amino acids; arginine, histidine, lysine; acidic amino acids: aspartic acid, glutamic acid; polar amino acids: asparagine, glutamine; small amino acids: alanine, serine, threonine, methionine, glycine; aromatic amino acids; phenylalanine, tryptophan, tyrosine, histidine; hydrophobic amino acids; leucine, isoleucine, valine. Cysteine is a special case, as it may usually be conservatively substituted with serine and any other polar uncharged, side chain and vice versa. Glycine may be used as substituent for any amino acid. Glycine can be substituted usually by a small side chain such as by alanine, serine, threonine. Proline may be usually substituted, or used as substituent for glycine.

In particular preferred embodiments the Fibroblast Growth. Factor 2 is a human Fibroblast Growth Factor 2, which is particularly useful for human administration. The sequence of human Fibroblast Growth Factor 2 may be of die amino acid sequence: MAAGSITTLPALPEDGGSGAFPPGHFKDPKRLYCKNGGFFLRIHPDGRVDGVREKSDPHI KLQLQAEERGYVSIKGVCANRYLAMKEDGRLLASKCVTDECFFFERLBSNNYNTYRSR KYTSWYYALKRTGQYKLOSKTGPGQKAILFLPMSAKS (SEQ ID NO: 7). The fragment of the human Fibroblast Growth Factor 2 may comprise the above mentioned substitutions, deletions or additions, in particular conserved substitutions. In particular preferred embodiments the inventive fragment comprises an amino acid sequence of up to R118, K119, Y120, T121, S122, W123, Y124, V125, A126, L127, K128, R129 or T130 of SEQ ID NO: 7. The fragment may further comprise a sequence beginning at G38 of SEQ ID NO:7 or any one of F39, F40, L41, R42, I43, H44, P45, D46, G47, R48, Y49, D50, G51, V52, R53, E54, K55, S56, D57, P58, H59, I60, K61, L62, Q63, L64, Q65, A66, E67, E68, R69, G70, Y71, Y72, S73, I74, K75, G76, Y77, C78, A79, N80, R81, Y82, L83, A84, M85, K86, E87, D88, G89, R90, L91, L92, A93, S94, K95, C96, V97, T98, D99, E100, C101, F102, F103 of SEQ ID NO; 7. In certain embodiments, the inventive fragment consists of these amino acids with optional further amino acids or peptides, but not of the Fibroblast Growth Factor 2. Such additional amino acids or peptides can, e.g., be of a pharmaceutically active substance.

In preferred embodiments, the inventive polypeptide fragment may comprise a length of at least 15, at least 16, at least 17, at least 18, at least 19, at least 20, at least 21, at least 22, at least 23, at least 24, at least 25, at least 26, at least 27, at least 28, at least 29, at least 30 amino acids. Furthermore, in combination with any of these lengths, the inventive polypeptide fragment of Fibroblast Growth Factor 2 may comprise up to 140, up to 130, up to 120, up to 110, up to 90, up to 80, up to 75, up to 70, up to 65, up to 60, up to 55, up to 52, up to 50, up to 45, up to 43, up to 40 amino acids of Fibroblast Growth Factor 2, optional with any of the above mentioned amino acid substitutions, deletions or additions (of a FGF2 or non-FGF2 sequence). The entire length of the polypeptide (including any optionally attached or fused additional amino acids may comprise up to 3000, 2500, 2200, 2000, 1800, 1500, 1200, 1000, 800, 700, 600, or up to 500 amino acids.

In special embodiments of the present invention, the polypeptide may comprise a label. Due to the selective binding of the inventive fragment to fibrin/fibrinogen, in particular for forming a depot at fibrin clots, visualization of regenerating areas with fibrin or fibrin clots within the patient is possible. This aspect is also useful to diagnose thrombosis. In further special embodiments a di- or multimeric form of the peptides can induce aggregation of fibrinogen monomers and lead to polymerisation of fibrinogen.

According to further aspects of the present invention pharmaceutical composition or kit comprising the polypeptide are provided. Such compositions or kits may be used for therapeutic application. Although the polypeptide is effective alone, the compositions or kits may comprise further pharmaceutically active substances.

According to a preferred embodiment of the present invention the polypeptide is designed for the incorporation in a “classical” tissue adhesive system. Such a system usually comprises a fibrinogen and a thrombin containing preparation similar to a “one-” or “two component” glue resulting in fibrin formation at the site of application or a preformed fibrin preparation, e.g. a fibrin fleece. The formed fibrin clot or the fibrin fleece allows e.g. wound closure or tissue adhesion. Further ingredients in this system are e.g. Factor XIII (as a cross-linker), fibrinolysis-inhibitors, etc.

In special embodiments of the present invention, the polypeptide fragment of the invention is conjugated to a pharmaceutically active substance, thus forming, a conjugate of the polypeptide with a pharmaceutically active substance. The inventive polypeptide may be used as a linker between fibrin and the pharmaceutically active substance. The conjugation of these two moieties can be done by any method known in the art, including chemical covalent or non-covalent binding such as by using linking agents, by recombinant methods, in particular expression as a fusion protein with or without amino acid linkers, or disulfide coupling. Example linking agents may further be capturing moieties that specifically recognizes and reversibly binds the pharmaceutically active substance of interest. Such a substance capturing moiety may be an antibody or a receptor or a part thereof such, as a F(ab) or F(ab)₂ part. Herein, the term “antibody” includes a complete antibody of any class, comprising the constant domain as well as the variable antigen binding domain, as well as parts of antibodies or antibody-derived molecules, e.g. fragments or recombinant constructs, which at least comprise an antigen binding domain that hinds the pharmaceutically active substance. The antibody may be monoclonal or polyclonal. Antibody binding can be specific, in particular a specificity expressed, in a high affinity constant for the pharmaceutically active substance of at least an affinity K_{A} of 1×10⁴ M⁻¹, 1×10⁵ M⁻¹, 1×10⁶ M⁻¹, 1×10⁷ M⁻¹ or higher affinity.

Another aspect of the present invention relates to hFGF-2 peptides as a binding moiety which binds to fibrinogen and fibrin and a substance capturing part capable binding to a pharmaceutically active substance. According to the present invention the hFGF-2 peptides are bound to the substance capturing moiety, preferably covalently. For example hFGF-2 peptides, which have a naturally affinity to fibrinogen and fibrin, may be hound or coupled to another active substance (i.e. proteins, peptides, organic molecules). This coupling may be accomplished by chemical linkers, by peptide synthesis or combination of these techniques. In addition, the described peptides can carry a tissue reactive group in the non-binding part of the peptide such as —NHS—, or other reactive chemical groups (e.g. —SH) or collagen binding sequences as described (see e.g. Guan J. et al. Biomaterials 2012, 33(5)p. 1386-965; Li X. et al., Biomaterials 2011, 32(32)p. 8172-81; Katikaneni R. et al. Int. J. Cancer 2011, Nov. 30 [Epub ahead of print])), antibodies or antibody fragments to tissue, components (e.g. extracellular matrix or cell membrane molecules).

The present invention may be adapted for all pharmaceutically active substances possible, especially for those for which a suitable binding partner is already known (e.g. antigen/anti body, receptor/ligand, complex partners). In each case, the binding partner to be applied as a drug is bound to the Conjugate according to the present invention only via its individual, corresponding binding partner, the latter being covalently coupled to the fibrin/fibrinogen-binding moiety.

Preferred pharmaceutically active substances to be used in the present conjugate or in a kit are antibiotics, growth factors, receptors for tissue components, tissue adhesive substances, anti-tumor agents, cell adhesive substances, nucleic acids, plasma proteins, anti-proteases, fibrinolysis-inhibitors, hormones, heparinoids, wound-healing substances tissue binding reacting sequences and mixtures thereof. When a pharmaceutically active substance is a fibrinolysis-inhibitor such as aprotinin, as part of the inventive conjugate, the dot to which the conjugate is bound will last longer than a clot which merely contains tree aprotinin, which would readily defuse out of the clot. Administering the inventive conjugate to a fibrin clot may form a drug depot of the pharmaceutically active substance. Preferred pharmaceutically active substances which are optimally fused to the polypeptide fragment are cytokines, growth factors and wound healing substances such as leptin, IL-8, MCP-1 and PF-4; antibiotic peptides such as magainins, defensins and granulysins; fibrinolysis-inhibitors such as aprotinin, eglin and Kunitz domains of human lipoprotein-associated coagulation-inhibitor or a lysine derivative blocking the plasminogen binding, such as e.g. tranexamic acid.

In particular preferred embodiments the inventive polypeptide is linked, to a protease-inhibitor, in particular to an inhibitor that increases stability of fibrin or prevents fibrin degradation. Special inhibitors are aprotinin or eglin. A further embodiment of the polypeptide according to the present invention may be linked to tissue binding reacting sequences or substances.

The pharmaceutically active substance thus released can serve to direct growth, migration and differentiation of specific cell types, thus enhancing wound healing and new vascularization during tissue repair even beyond the capabilities of the inventive fragment, if used alone.

The fibrinogen and fibrin binding domain of hFGF-2 is located in the third quarter of the amino acid sequence of hFGF-2. Peptides from hFGF-2 in this binding domain area can be selected and recombinantly or chemically synthesized. Therefore, the present invention also relates to methods of producing the inventive polypeptide fragments, either by expression in a suitable expression system (such as E. coli) or synthesis.

Peptides from hFGF-2 according to the present invention may be recombinantly produced using the pGEX vector system, which contains a sequence for N-terminal glutathione S-transferase (GST), which results in a fusion protein of GST and the protein of interest. After the expression in E. coli the GST-tag can be cleaved from the fusion protein. Such a cleavage site that may be used is e.g. the PreScision protease site.

Usually, small human FGF-2 peptides according to the present invention are synthesized chemically, e.g. fragments that are smaller than 32 amino acids. In a further aspect of the invention, a DNA molecule is provided that encodes a polypeptide of the invention. The DNA molecule may comprise a suitable promoter for expression in a host cell. Preferably, the DNA molecule is a vector, especially an expression vector.

In a further aspect of the present invention, the polypeptide may be used in a therapy. Such a therapy may be associated with the deposition of fibrin, in particular comprising a blood clotting reaction, a fibrin(ogen) aggregation reaction and/or a fibrin(ogen) solidification reaction. Particular preferred therapies are for wound healing and/or hemostasis. A therapy may comprise administration of the polypeptide to a subject, optionally together with the pharmaceutically active substance, with or without conjugation to the polypeptide.

Usually, the polypeptide is administered as part of a pharmaceutical composition. Such a pharmaceutical composition my comprise a buffer, tonic substance, stabilizer, protease-inhibitor or carrier as known in the art. In particular preferred embodiments, the composition may further comprise fibrin or fibrinogen.

The composition may comprise buffer substances or tonic substances. By means of a buffer, the pH of the composition can be adjusted to physiological conditions, and, moreover, pH fluctuations can be attenuated or buffered. An example thereof is a phosphate buffer. Tonic substances are for adjusting the osmolality and may comprise ionic substances, such as e.g. inorganic salts, such as NaCl, or also non-ionic substances, such as e.g. glycerol or carbohydrates.

Pharmaceutical carrier substances serve for a better tolerance of the composition and allow for better solubility as well as better bioavailability of the active substances contained in the composition. Examples of this are emulsifiers, thickening agents, redox components, starch, alcohol solutions, polyethylene glycol or lipids. The choice of a suitable pharmaceutical carrier is highly dependent on the manner of administration. E.g. for injections, liquid final compositions are requited.

The composition may be used or prepared for an intravenous, intra-arterial, intramuscular, intravascular, intraperitoneal or subcutaneous administration. For these purposes, e.g. injections or transfusions are suitable. Administrations directly into the blood stream have the advantage that the active substances of the composition will be distributed in the entire body and will quickly reach the target tissue or site of a fibrin clot.

In an preferred embodiment, the inventive polypeptide is used in a method for improving wound healing and/or hemostasis in a subject, comprising administering the polypeptide to said subject. The present invention also relates to a method of inducing cell migration to a fibrin clot or increasing cell differentiation or proliferation at the location of a fibrin clot, comprising administering a polypeptide to said clot. The invention further provides the method of localizing a pharmaceutically active substance to a fibrin clot, comprising administering a conjugate of the polypeptide with said pharmaceutically active substance to said clot. The described peptides may also be provided in a di or multimeric form to form hydrogels with fibrinogen.

The components of the inventive composition may also be provided in a kit, in particular a kit comprising the polypeptide and a pharmaceutically active substance as described above. The polypeptide may further comprise a linking moiety or a substance capturing moiety such as an antibody, as described above. The kit may further comprise fibrin or fibrinogen in preferred embodiments.

According to another aspect the present invention relates to a kit for forming a depot for a pharmaceutically active substance comprising a tissue adhesive based on fibrinogen and a polypeptide according to the present invention. The polypeptide may be provided in a separate form ready to be mixed before medical use. The “ready to use” mixture of the tissue adhesive based on fibrinogen and the polypeptide according to the present invention may be applied with means and methods as already known in the art for “classical” tissue adhesives, especially with the fibrinogen component of such adhesives. This fibrinogen component may be mixed in a known way with a component containing an activity for processing fibrinogen to fibrin, preferably a thrombin preparation or with a di or multimeric human FGF peptide to induce hydrogel formation.

A kit according to the present invention may therefore also contain suitable devices for administering the tissue adhesive and the conjugate and optionally the fibrinogen to fibrin processing activity. Examples for such devices are described in EP 0 037 393 A, EP 0 315 222 A, EP 0 156 098 A, EP 0 210 160 A and EP 0 292 472 A, which are incorporated herein by reference.

The invention will now be explained in more detail by way of examples and the drawing figures to which, however, it shall not be restricted.

### Example 1

### a) Construction of hFGF-2 and Various Truncated hFGF-2 Expression Plasmids

Using a standard PCR program (95° C., 30 sec; 60° C., 30 sec; 72° C., 30 sec; 25 cycles), full length hFGF-2 cDNA was amplified with primers containing EcoRI and XhoI restriction sites and a HIS-tag (6×) on the C-terminus (Table 1).

TABLE 1
Primers containing EcoRI and XhoI restriction sites were used for
subcloning full length hFGF-2 cDNA and hFGF-2 cDNA fragments into pGEX-6P-2
expression vector. The restriction sites are inderlined and the 6x HIS-tag
on the C-terminus is marked in italics. The sequences of the synthetic
peptides #3, #4, and #5 contain a 5x HIS-tag at the C-terminus (italics).

| Primers | sense (s) and antisense (as); 5′-3′ |
|---|---|
| EcoR I restriction site | |
| hbFGF full length sense | GGA ATT CCC ATG GCA GCC GGG AGC ATC (SEQ ID NO: 8) |
| hbFGF pep1 sense | GGA ATT CCC GAA GAG AGA GGA GTT GTG (SEQ ID NO: 9) |
| hbFGF pep2 sense | GGA ATT CCC GTG TGT GCT AAC CGT TAC (SEQ ID NO: 10) |
| Xho I restriction site | |
| hbFGF full length | CTC GAG TCA ATG ATG ATG ATG ATG ATG GCT CTT AGC |
| antisense | AGA CAT TGG (SEQ ID NO: 11) |
| hbFGF pep antisense | CTC GAG TCA ATG ATG ATG ATG ATG ATG CCT TGA CCG |
| | GTA AGT ATT (SEQ ID NO: 12) |
| Synthetic peptides | |
| pep 3 (37 aa) | EDGRLLASKC VTDECFFFER LESNNYNTYR SRHHHHH (SEQ ID NO: 13) |
| pep 4 (29 aa) | KCVTDECFFF ERLESNNYNT YRSRHHHHH (SEQ ID NO: 14) |
| pep 5 (20 aa) | FERLESNNYN TYRSRHHHHH (SEQ ID NO: 15) |

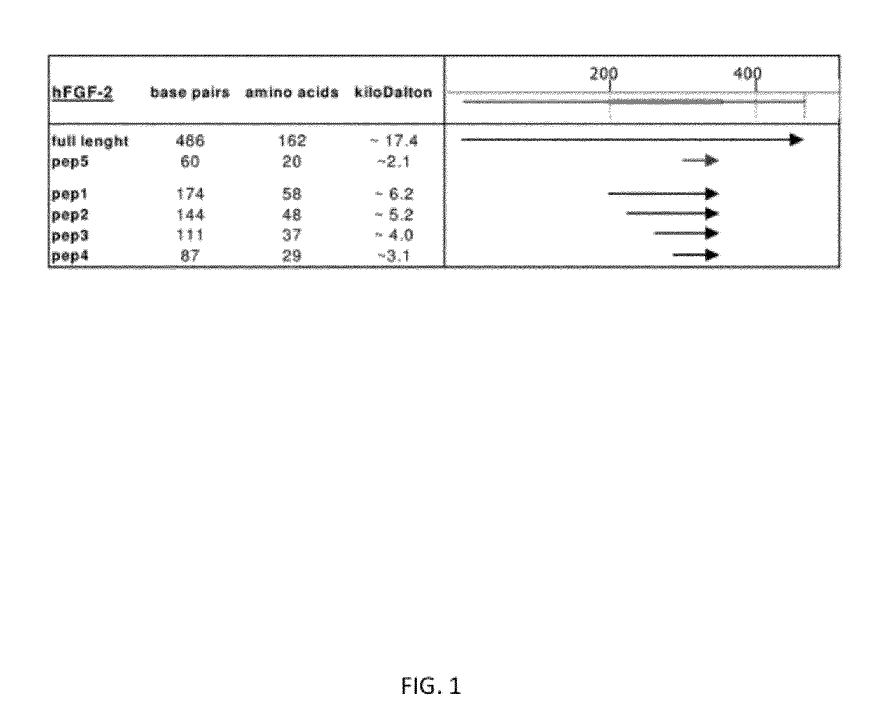

PCR products (FIG. 1), ranging from 486 bp down to 111 bp were digested with EcoRI and XhoI and then ligated into the pGEX-6P-2 expression vector (Pharmacia Biotech, Vienna) using DNA Ligation Kit (Roche Diagnostics. Vienna). The pGEX vector system contains a sequence for N-terminal glutathione S-transferase (GST), which results in a fusion protein of GST and the protein of interest. It also includes a PreScision protease site to cleave the GSTtag from the fusion protein. After transformation into Escherichia coli Top 10 (Invitrogen, Germany), plasmid DNA of positive clones were isolated and purified (Mini Preparation Kit, Sigma-Aldrich, Vienna) and finally sequenced (Boehringer Ingelheim Austria GmbH, Vienna).

### b) Expression and Purification

Plasmids containing the correct fragment sequences were transformed into Escherichia coli BL21(DE3) (invitrogen, Germany). Protein expression was induced with 1 mM isopropyl-b-D-thiogalactopyranoside (IPTG). After 5 hours on room temperature cells were harvested and pellets were resuspended in phosphate buffered saline (PBS, pH 7.5), 5 mM 1,4-dithio-DL-threitol (DTT), 1% Triton X-100, protease inhibitor cocktail (Roche Diagnostics, Vienna), and 0.5 μg/ml lysozyme. Bacteria were incubated for 15 minutes on ice, sonicated 6 times for 10 seconds and the lysates were centrifuged for 15 minutes. Supernatants were then incubated with 10% glutathione Sepharose slurry (Pharmacia Biotech, Vienna) over night at 4° C. Afterwards the column was washed once with lysis buffer and twice with cleavage buffer (50 mM Tris-HCl, pH 7.0; 150 mM NaCl; 0.01% Triton X-100), GST fusion proteins were cleaved with Precision protease (Amersham Biosciences, Germany) over night at 4° C. and hFGF-2 peptides were eluted using 50 mM Tris-HCl (pH 7.0) and 10 mM reduced glutathione. Finally, purified proteins were dialyzed against PBS using a minidialysis system (Fierce, THP Medical Products, Vienna) and stored at −80° C.

### c) SDS-PAGE and Western Blot Analysis

SDS-PAGE (sodium dodecylsulfate-polyacrylamide gel electrophoresis) followed by Coomassie brilliant blue staining, was performed at each step of the recombinant protein as previously described. Peptides (<7 kD) were separated, by discontinuous tricine SDS-PAGE according to manufacturer's instruction (Bio-Rad Laboratories, Vienna). For Western blot analysis, proteins were electrophoretically transferred to a nitrocellulose membrane (Bio-Rad, Vienna). The membrane was first immersed in a blocking solution (PBS and 2% bovine serum albumin (BSA)) and then incubated for 1 hour with a monoclonal anti-His6-peroxidase conjugate (1:1.000) in PBS containing 0.2% TWEEN (PBST 0.2). After washing three times (10 minutes) with PBST 0.2, the membrane was covered with a chemiluminescent detecting reagent (Roche Diagnostics, Vienna). Immuno-reactive bands were visualized by the Multiimage™ Light Cabinet (Biozym, Vienna).

### d) Synthetic Peptides

Due to the small sizes and therefore difficult expression peptides pep3 (37 aa), pep4 (29 aa) and pep5 (20 aa) were synthesized by a company (piCHEM, Graz). The sequences are shown in Table 1.

### e) Biological Activity of Recombinant hFGF-2 and Peptides

A moose myoblast cell line (C2C12; ECACC (Sigma-Aldrich, Vienna)) was grown in Dulbecco's Modified Eagle Medium (DMEM) with 10% fetal calf serum (FCS). At 50-60% confluence cells were trypsinized and transferred to a 24-well plate (1×10⁴/well).

Following a 48-hour-culture period at 37° C., cells were washed with DMEM only and then covered with medium (DMEM+1% FCS) containing various concentrations of hFGF-2 or hFGF-2 peptides. After 48 hours number of viable cells was determined by adding 3-(4,5-dimethylthiazol-2-yl)-2,5-diphenyltetrazolium bromide (MTT, Sigma-Aldrich, Vienna) as previously described {M05}.

### f) Fibrin(ogen) Binding Assay by ELISA and SPR

A polystyrene 96-well plate (Nunc® Maxisorp EUSA-plates) was coated with 1 mg/ml fibrinogen (Tisseel®, Baxter AG, Vienna) overnight at 4° C. and then blocked with blocking solution (PBS+1% BSA) for 2 hours at room temperature CRT). After washing three times with PBS, purified peptides were added in various dilutions to the plate and incubated for 1 hour at RT. After the three times washing with PBS, a monoclonal anti-His6-peroxidase antibody (Sigma-Aldrich, Vienna), diluted in PBS+0.1% Tween (Sigma-Aldrich, Vienna) was added and incubated for 1 hour at RT. Finally, the plate was washed three times with PBS again and wells were incubated with o-phenylenediaminehydrochlorid substrate. Absorbance was measured at 492 nm and 620 nm. For fibrin binding analysis, the plate was first coated with fibrinogen overnight at 4° C. and then incubated with 10 I.U./ml thrombin (Tisseel, Baxter AG, Vienna) for 30 minutes at 37° C. The following procedure was the same as described above. For the surface plasmon resonance (SPR) method fibrinogen was coupled to a gold sensor chip (10% COOH—(PEG)₆-C₁₁—SH, 90% OH(PEG)₃-C₁₁—SH; Reichert Analytical, NY, USA) by amine chemistry using EDC (1-ethyl-3-[3-dimethylaminopropyl]carbodiimide hydrochloride) and NHS (N-hydroxysuccinimide) (Pierce, Ill., USA). Uncoupled sites on the gold sensor chip were blocked with 1 M ethanolamine pH 8.5 (Sigma-Aldrich, MO, USA). The purified recombinant proteins and peptides were passed over the chip's surface at a flow rate of 0.4 ml/min to 0.8 ml/min and allowed to interact. The signals were detected and recorded at the Reichert SR7000 Surface Plasmon Resonance Analytical Instrument (Reichert Analytical, NY, USA). After each measurement the sensor chip surface was regenerated by a pulse with 100 mM phosphoric acid (Mallinckrodt Chemicals, NJ, USA) after each application of the samples. Association and dissociation signals were fitted to one association curve using a prism software calculation program.

Statistical, evaluations were performed using ANOVA with p<0.05 as being significant. All values are expressed as means±standard deviation (S.D.).

### g) Visualization

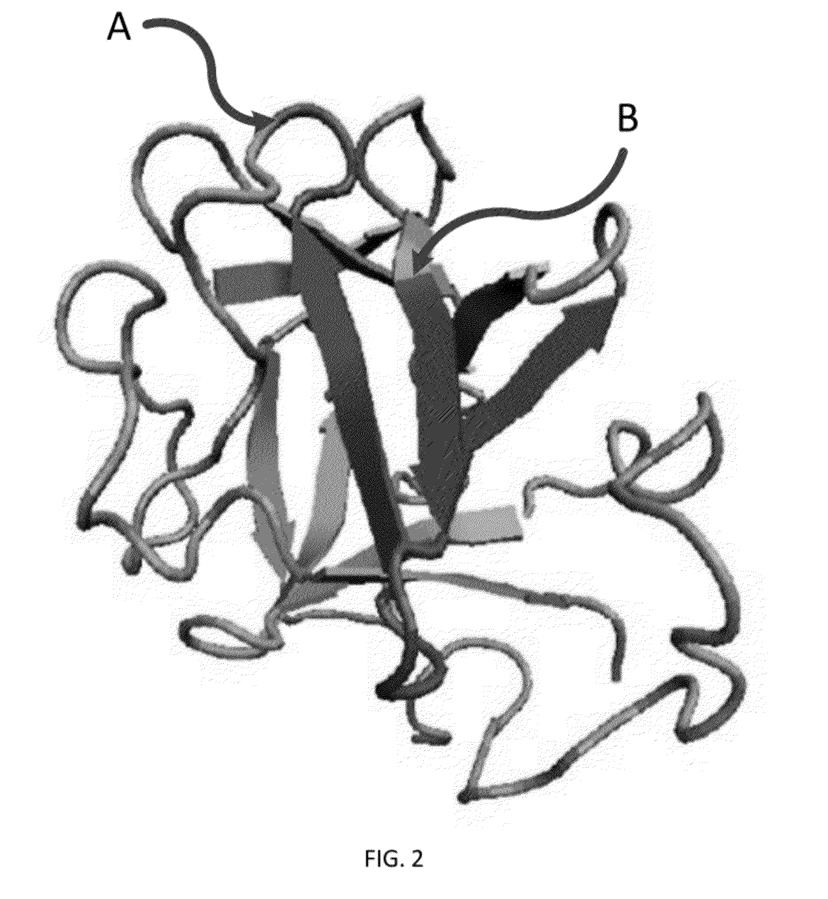

For structure visualization of human FGF-2 and the putative binding domain the software program Visual Molecular Dynamics was used. The structure visualization showed a well accessible protein domain of hFGF-2 for naturally binding partners (FIG. 2).

### h) Expression and Purification of hFGF-2 Peptides and hFGF-2

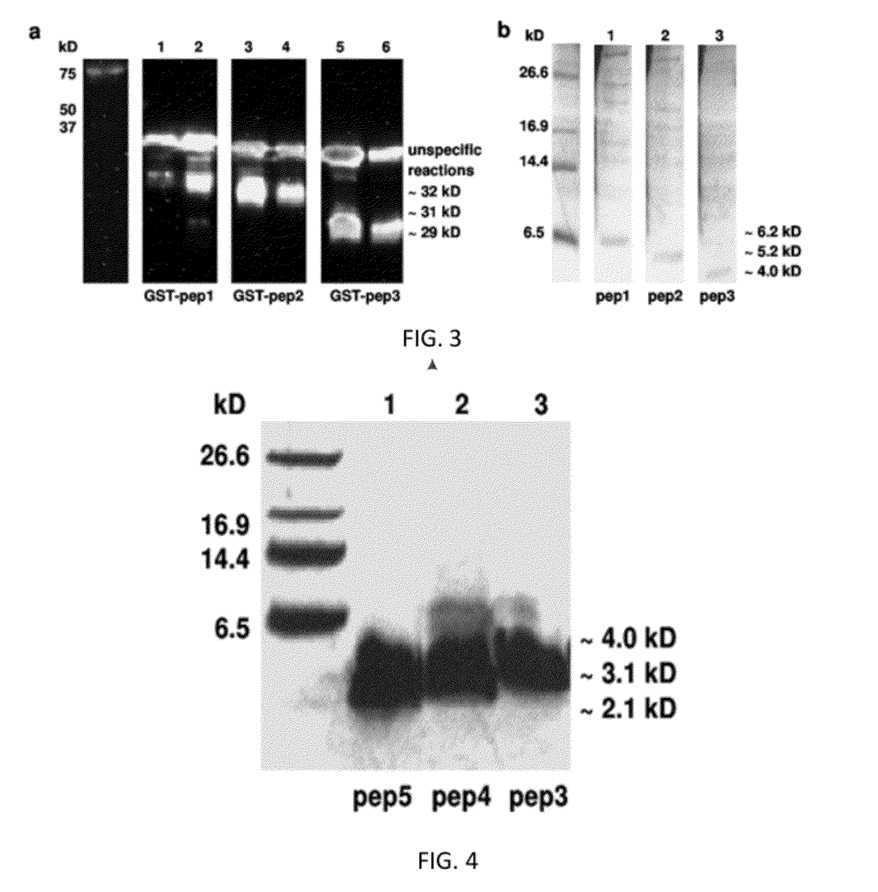

Amino- and carboxyl-terminally truncated hFGF-2 peptides (pep1 to pep3) were successfully expressed as GST fusion proteins (FIG. 3a). Western blot analysis represented similar protein amounts in both, supernatant and insoluble fraction. Purified peptides pep1 (˜32 kD), pep2 (˜5.2 kD), and pep3 G-29 kD) were analyzed by tricine SDS-PAGE (FIG. 3b). Fragments smaller than 37 amino acids were chemically synthesized and the different sizes of the peptides pep3, pep4, and pep5 were confirmed (FIG. 4). Additionally, recombinant hFGF-2 was also expressed as a fusion protein using the pGEX-6P-2 expression system. Western blot technique revealed a high amount of correctly sized protein in the supernatant (data not shown).

### i) Binding Assay

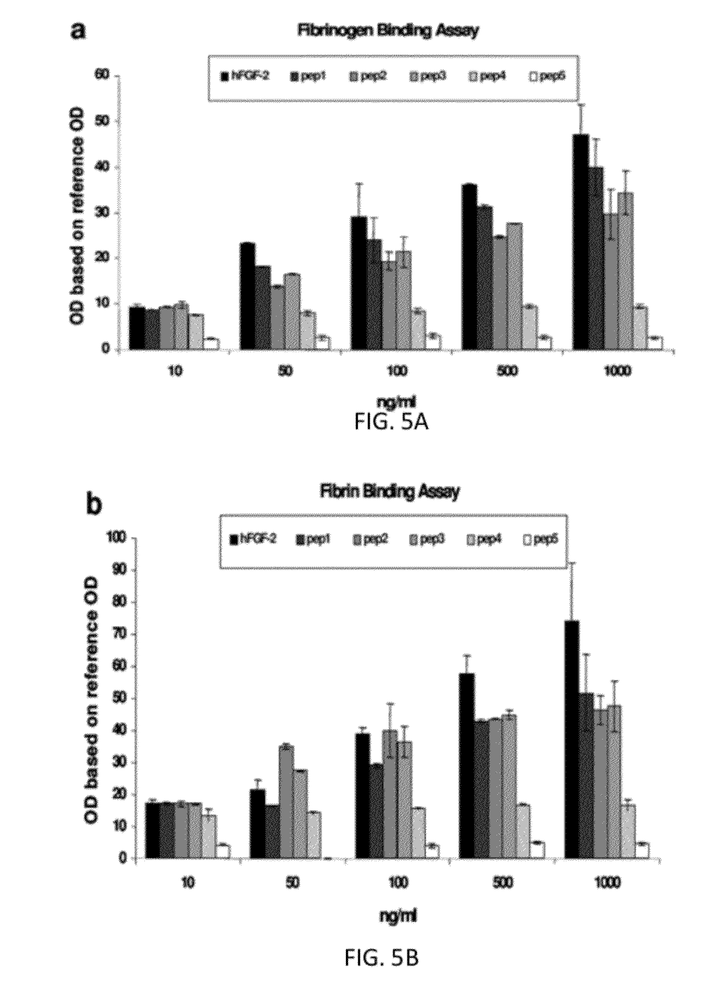

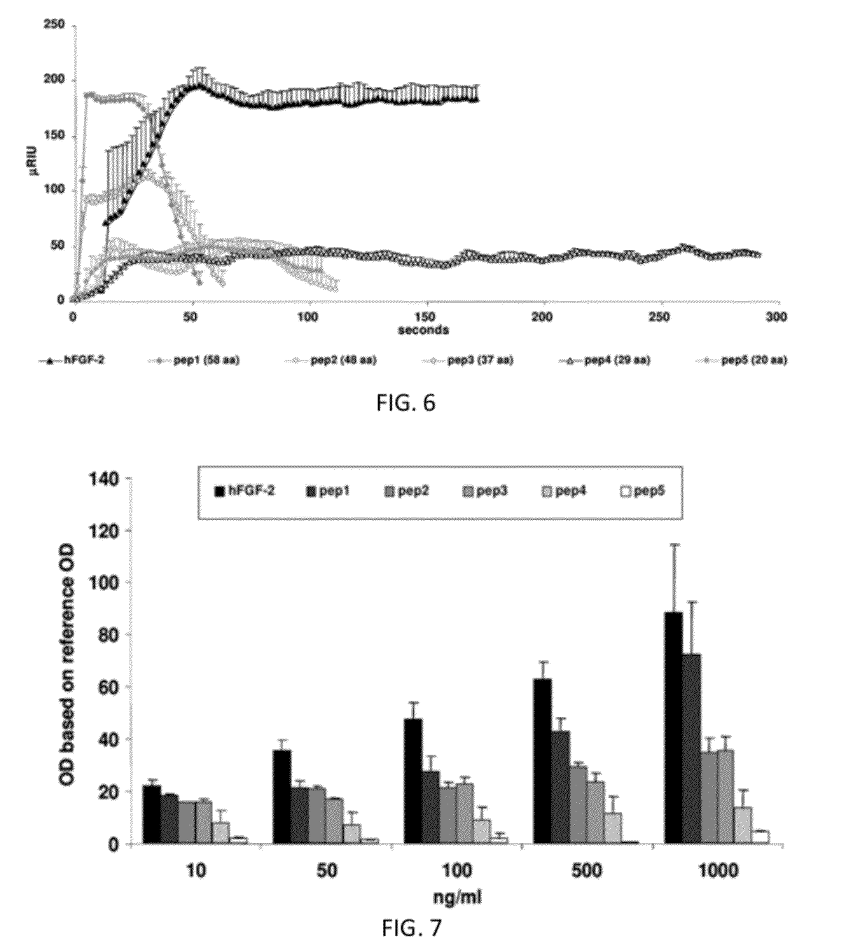

Purified, proteins and peptides in different concentrations were used for testing the binding affinity to fibrinogen and fibrin (FIG. 5). The results of the fibrinogen and fibrin binding assay demonstrated a high affinity of native rhFGF-2, pep1, pep2, and pep3 to fibrinogen. Peptide pep4 showed a significantly lower binding and pep5 has nearly no binding affinity in various concentrations. Surface plasmon resonance experiments showed peptide pep4 with similar binding characteristics to fibrinogen as the full-length recombinant hFGF-2 (FIG. 6). The binding signal of pep4 was significantly lower compared to hFGF-2 but more consistently over a longer period of 300 seconds. Peptides pep1, pep2, pep 3 and pep5 showed weak and short binding signals.

### j) Biological Activity

Recombinant hFGF-2 showed the highest proliferative activity in mouse myoblast cells. Pep1 to pep3 promoted cell proliferation almost to the same extent. The smallest fragment pep5 did not stimulate C2C12 cell growth (FIG. 7).

### Example 2

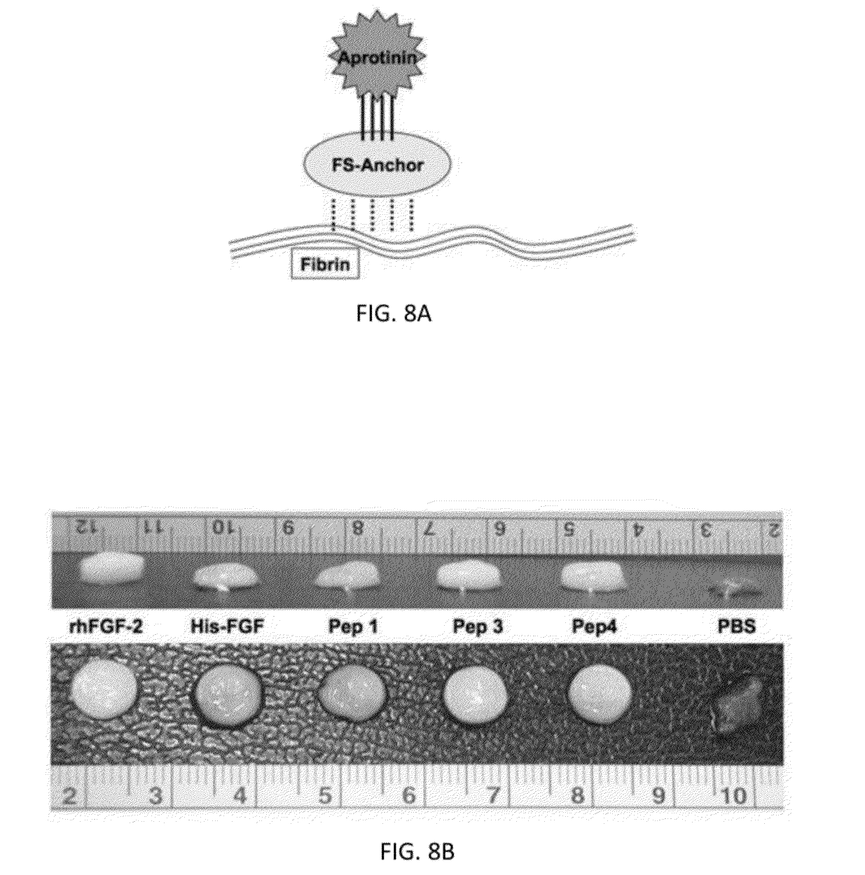

Aprotinin was successfully covalently bound to one FS-anchor (His-FGF-2, pep1 (68 an), pep3 (37 aa) and pep4 (28 aa)) using a standard protocol for 1-ethyl-3-(3-dimethylaminopropyl)carbodiimide) (EDC) coupling (FIG. 8A). For a positive control recombinant human FGF-2 (ProSpec Tany, Israel) and for a negative control phosphate buffered saline (PBS) were used. Covalently linked to a FS-anchor and purified aprotinin was mixed with fibrinogen and finally with thrombin to form fibrin clots. These fibrin clots were kept in PBS at 37° C. to observe stability and degradation of fibrin. After 5 days fibrin clots with the pep3 and pep4 FS-anchor showed a higher stability as die fibrin clots with His-FGF-2 and pep1 (FIG. 5B).

### Discussion

It was the aim to produce biological active hFGF-2 peptides with a binding affinity to fibrinogen and fibrin. hFGF-2 peptides as well as foil length hFGF-2 were recombinantly expressed and purified. Human FGF-2 is a single chain polypeptide without any disulfide bonds and glycosylation. Therefore, the FGF-2 peptides aid FGF-2 are suitable to be expressed in E. coli. Amplified hFGF-2 cDNA fragments and full length hFGF-2 cDNA were ligated into pGEX-6P-2 vector which contains a GST-lag at the N-terminus. The GST-fusion proteins led to a higher expression rate of hFGF-2 than direct expression (e.g. pET-11a expression system) of hFGF-2.

Furthermore, this system permits convenient site-specific cleavage and simultaneous purification on Glutathione Sepharose™. The high expression rate of a N-terminal fused protein and the following simultaneous purification and cleavage of the protein of interest allows the production of the hFGF-2 peptides pep1-pep3. Consequently, to eliminate non specific reactions for the binding assays to fibrinogen and fibrin peptides pep3, pep4, and pep5 were all chemically synthesized.

The results of the binding assay demonstrate that all peptides with exception of pep4 had binding affinity to both fibrinogen and fibrin. Pep5 (20 aa) showed little binding affinity to both fibrinogen and fibrin. Surface plasmon resonance results confirmed the binding affinity to fibrinogen especially for pep4 (29 amino acids). The fitted association curve of this peptide showed lower binding signals than that of hFGF-2 but similar association and dissociation rates Compared to pep4 and full-length hFGF-2 the SPR of the other peptides resulted in weak and unstable binding signals. Based on these results, the fragment preferably comprises more than 15 amino acids of pep5 for the binding of hFGF-2 to fibrinogen. The extension can e.g. be on the N-terminal site such as by 4 amino acids (pep4).

In addition to fibrin/fibrinogen binding, the fragments had to retain biological activity. The results demonstrate a concentration dependent biological activity of hFGF-2 and the hFGF-2 peptides pep1 to pep5. Pep4 showed less activity, but similar to full length hFGF-2. Full length hFGF-2 showed the highest cell growth effect. Pep2 and pep3 retained approximately 50% of the activity of the full length protein. The smallest peptide (pep5) possesses little biological activity. Consequently, the results indicate that the domain for biological activity and the binding domain to fibrinogen are located on the same segment of hFGF-2 gene. Peptide pep4 with 37 aa and a molecular weight of about 4 kD showed similar binding characteristics as foil length hFGF-2. Therefore, it is possible that hFGF-2 fragment can be used on its own or in a further aspect as a linking agent between fibrinogen and target substances. That could lead to a continuous slow-release out of the fibrin matrix. If used by itself, the biological activity of these peptides could stimulate cell proliferation and/or cell differentiation and thereby have beneficial effects on angiogenesis and wound healing and/or hemostasis.

All patents, patent publications, patent applications, journal articles, hooks, technical references, and the like discussed in the instant disclosure are incorporated herein by reference in their entirety for all purposes.

While the above provides a full and complete disclosure of exemplary embodiments of the present invention, various modifications, alternate constructions and equivalents may be employed as desired. Consequently, although the embodiments have been described in some detail, by way of example and for clarity of understanding, a variety of modifications, changes, and adaptations will be obvious to those of skill in the art. Accordingly, the above description and illustrations should not be construed as limiting the invention, which can be defined by the claims.

### REFERENCES

1. Peng, H., Salmi, A., Fay, P., Bellum, S., Prudovsky, I., Maciag, T., and Francis, C. W. Identification of a binding site on human FGF-2 for fibrinogen. Blood, 103: 2114-2120, 2004.
2. Sahni, A. and Francis, C. W, Vascular endothelial growth factor binds to fibrinogen and fibrin, and stimulates endothelial cell proliferation. Blood, 96: 3772-3778, 2000.
3. Tonnesen, M. G., Feng, X., and Clark, R. A. Angiogenesis in wound healing. J Investig Dermatol Symp Proc, 5: 40-46, 2000.
4. Sahni, A., Guo, M., Sahni, S. K., and Francis, C. W. Interleukin-1beta but not IL-1alpha binds to fibrinogen and fibrin, and has enhanced activity in the bound form. Blood, 104: 409-414, 2004.
5. Gospodarowicz, D., Ferrara, N., Schweigerer, L., and Neufeld, G. Structural characterization and biological functions of fibroblast growth factor. Endocr Rev. 8: 95-114, 1987.
6. Florkiewicz, R. Z., Shibata, F., Barankiewicz, T., Baird, A., Gonzalez, A. M., Florkiewicz, E., and Shah, N. Basic fibroblast growth factor gene expression. Ann N Y Acad Sci, 638: 109-126, 1991.
7. Shibata, P., Baird, A., and Florkiewicz, R, Z. Functional characterization of the human basic fibroblast growth factor gene promoter. Growth Factors, 4; 277-287, 1991,
8. Bugler, B., Amalric, F., and Prats, H. Alternative initiation of translation determines cytoplasmic or nuclear localization of basic fibroblast growth factor. Mol Cell Biol, 11; 573-577, 1991.
9. Florkiewicz, R. Z. and Sommer, A. Human basic fibroblast growth factor gene encodes four polypeptides: three initiate translation from non-AUG codons. Proc Natl Acad Sci U S A, 86: 3978-3981, 1989.
10. Zhang, J. D., Cousens, L, S., Barr, P. J., and Sprang, S. R. Three-dimensional structure of human basic fibroblast growth, factor, a structural homolog of interleukin 1 beta. Proc Natl Acad Sci USA, 88; 3446-3450, 1991.
11. Gospodarowicz, D. Biological activities of fibroblast growth, factors. Ann N Y Acad Sci, 638: 1-8, 1991.
12. Folkman, J. Anti-angiogenesis: new concept for therapy of solid tumors. Ann Surg, 175: 409-416, 1972.
13. Sahni, A., Odrljin, T., and Francis, C. W. Binding of basic fibroblast growth factor to fibrinogen and fibrin. J Biol Chem, 273: 7554-7559, 1998.
14. Jackson. M. R. Fibrin sealants in surgical practice: An overview. Am J Surg, 182: 1S-7S, 2001.
15. Mittermayr, R., Wassermann, E., Thurnher, M., Simunek, M., and Redl, H. Skin graft fixation by slow clotting fibrin sealant applied as a dun layer. Burns, 32; 305-311, 2006.
16. Sarpel, U., Roayaie, S., Schwartz, M. E., and Labow, D. M. The role of fibrin sealants in hepatic surgery. Surg Technol Int, 16: 31-36, 2007.
17. Newton, C., Goodwin, C., Helgerson, S., and Spaethe, R. Human Growth Factors in the Fibrinogen Complex Component of Fibrin Sealant Products. World Biomaterials Congress, Australia. 2004.
18. Morton, T. J., burst, W., van Griensven, M., and Redl, H. Controlled release of substances bound to fibrin-anchors or of DNA. Drug Deliv, 16: 102-107, 2009.

1. A polypeptide fragment of Fibroblast Growth Factor 2, wherein said fragment binds to fibrinogen or to fibrin and/or increases cell proliferation, differentiation or migration.

2. The polypeptide according to claim 1, wherein said fragment promotes wound healing.

3. The polypeptide according to claim 1, wherein said fragment is conjugated to a pharmaceutically active substance.

4. The polypeptide according to claim 3, wherein the fragment is conjugated to a pharmaceutically active substance by a linking agent.

5. The polypeptide according to claim 4, wherein the linking agent is selected from the group of carbodiimides, such as 1-ethyl-3-(3-dimethyl-aminopropyl)carbodiimide (EDC), bis-diazotized benzidine (BDB), a bifunctional glutaraldehyde, a heterobifunctional reagent such as m-maleimidobenzoyl-N-hydroxysuccinimide ester (MBS), SH coupling.

6. The polypeptide according to claim 1, which has essentially the biological activity as fibroblast growth factor 2 on cell proliferation, differentiation or migration.

7. The polypeptide according to claim 1, wherein said polypeptide increases proliferation of a fibroblast cell, a myoblast cell, an endothelial cell, a stem cell, or any combination thereof.

8. The polypeptide according to claim 1 comprising an amino acid sequence of:
EERGVVSIKG VCANRYLAMK EDGRLLASKC VTDECFFFER LESNNYNTYR SR (SEQ ID NO:1),
GVCANRYLAM KEDGRLLASK CVTDECFFFE RLESNNYNTY RSR (SEQ ID NO:2),
EDGRLLASKC VTDECFFFER LESNNYNTYR SR (SEQ ID NO:3),
LLASKCVTDE CFFFERLESN NYNTYRSR (SEQ ID NO:4),
KCVTDECFFF ERLESNNYNT YRSR (SEQ ID NO:5), or
FERLESNNYN TYRSR (SEQ ID NO:6),
with optionally 1 to 10 amino acid substitutions or deletions.

9. The polypeptide of claim 8, wherein the substitutions are conserved substitutions.

10. The polypeptide according to claim 1, wherein said Fibroblast Growth Factor 2 is a human Fibroblast Growth Factor 2.

11. The polypeptide according to claim 10, wherein said Fibroblast Growth Factor 2 comprises the amino acid sequence MAAGSITTLPALPEDGGSGAFPPGHFKDPKRLYCKNGGFFLRIHPDGRVDGVREKSDPHI KLQLQAEERGVVSIKGVCANRYLAMKEDGRLLASKCVTDECFFFERLESNNYNTYRSR KYTSWYVALKRTGQYKLGSKTGPGQKVULFLPMSAKS (SEQ ID NO: 7).

12. The polypeptide according to claim 1 comprising a length of at least 20 amino acids.

13. The polypeptide according to claim 1 consisting of up to 140 amino acids.

14. The polypeptide according to claim 1 comprising a label.

15. The polypeptide according to claim 1 linked to a protease inhibitor or a lysine derivative.

16. The polypeptide according to claim 15 wherein said protease inhibitor increases stability of fibrin or prevents fibrin degradation.

17. The polypeptide according to claim 15 wherein said inhibitor is aprotinin or eglin.

18. The polypeptide according to claim 1 linked to tissue binding reacting sequences.

19. The polypeptide according to claim 1 adapted to crosslink fibrinogen or fibrin chains.

20. (canceled)

21. (canceled)

22. (canceled)

23. A pharmaceutical composition comprising a polypeptide according to claim 1.

24. (canceled)

25. The composition of claim 23 further comprising fibrin or fibrinogen.

26. The composition of claim 23, further comprising fibrin and a pharmaceutically active substance, wherein the polypeptide is a linker between fibrin and the pharmaceutically active substance.

27. A method of improving wound healing and/or hemostasis in a subject comprising administering a polypeptide according to claim 1 to said subject.

28. (canceled)

29. (canceled)

30. (canceled)

31. (canceled)
